(19) United States
(12) Patent Application Publication
(10) Pub. No.: US 2026/0007814 A1
(43) Pub. Date: Jan. 8, 2026

(54) VENOUS ACCESS DISLODGEMENT DETECTION SYSTEM AND METHOD

(71) Applicant: Outset Medical, Inc., San Jose, CA (US)

(72) Inventor: Michael HOGARD, San Jose, CA (US)

(21) Appl. No.: 18/766,615

(22) Filed: Jul. 8, 2024

(51) Int. Cl. A61M 1/36

(52) U.S. Cl. CPC A61M 1/3656

(57) ABSTRACT

A venous needle dislodgment detection system for use with a blood access device during medical treatment is disclosed, comprising a medical blood access system in communication with a dislodgment sensing apparatus, the medical blood access system having a venous blood line; a first end of the venous blood line being coupled to the medical blood access system at a tubing connection point, and a second end of the venous blood line being coupled to the blood access device; the dislodgment sensing apparatus comprising a sensor positioned adjacent to the venous blood line and proximal to the tubing connection point, the dislodgment sensing apparatus being configured to generate a dislodgment signal in response to detecting a displacement force indicative of a potential dislodgment of the blood access device from the patient; and the medical blood access system being configured to automatically generate an alarm condition in response to receiving the dislodgment signal.

### TECHNICAL FIELD

The present disclosure relates generally to medical treatments. More particularly, the present disclosure relates to the detection of needle dislodgement from a patient during medical treatments, therapies, or procedures involving access to the patient's circulatory system.

### BACKGROUND

Various medical therapies and procedures require accessing a patient's circulatory system to remove blood from a patient. During hemodialysis therapy, for example, an extracorporeal circuit is used to remove waste, toxins, and excess water from a patient's blood. In particular, blood is removed from a patient's vascular access through an arterial blood line, pumped through the blood-side of a dialyzer, and returned to the patient's vascular access through a venous blood line. Unless detected quickly, the patient can suffer a significant and potentially catastrophic blood loss if the venous needle becomes dislodged from the patient's vascular access during therapy. Such venous needle dislodgment events (“VND events”) refers generally to the disconnection of a needle, catheter, or other interface to the patient's vascular access.

One method of monitoring for such VND events includes venous and arterial pressure monitoring. For example, when the venous needle disconnects, the backpressure from the patient's access is lost, which can be detected as a drop in the venous pressure on a dialysis machine. However, much of the venous pressure signal is composed of the flow resistance through the access needle (which is not lost when the needle is dislodged), and thus the relative change in venous pressure from a disconnection event is very small. Because other actions (e.g., repositioning of the patient's arm) can also produce blood pressure fluctuations in this range, this type of pressure monitoring cannot reliably detect VND events. Other existing methods of detecting VND events include specialized moisture detection bandages or sensors placed at the vascular access site, and electrical continuity monitors designed to detect the physical disconnection of the venous needle from the patient's body.

Due to the extreme risks, it is of the utmost importance that VND events be detected quickly, accurately, and reliably. Although the existing measures do provide some ability to detect VND events, none of the existing methods or systems have proven sufficiently reliable or cost-effective. Further, while the risk of an undetected VND event exists even in clinical settings, patients undergoing dialysis treatment at home are at even greater risk of bleeding to death due to an undetected VND event.

The disclosed system and method are directed to overcoming one or more of the problems set forth above and/or other problems of the prior art.

### SUMMARY

In one aspect, the present disclosure is directed to a device for the detection of VND events. The system may include a physical or optical sensor configured to detect a force placed upon a venous blood line at the treatment console. A force of as little as about two pounds may be enough to cause a venous needle to be removed from a patent's cannulation site when held in place by a single piece of medical tape. Since such a minimal amount taping would below industry standards, detecting such a degree of force may allow for the prevention of a VND event. The detection of additional force in excess thereof placed upon a venous blood line may further pause treatment to allow for the evaluation of the injection site.

In another aspect, the present disclosure is directed to a method for the detection of VND events while performing dialysis. This method may include the detection of a force indicative of a VND event, triggering a warning indicative of such an event, pausing treatment in response to such event, and allowing for the reinstitution of treatment within set limits.

### BRIEF DESCRIPTION OF THE DRAWINGS

FIG. 1 is an isometric illustration of an exemplary treatment environment.

FIG. 2 is a diagrammatic illustration of an exemplary disclosed system for detecting a venous needle dislodgement in the environment of FIG. 1.

FIG. 3 is an isometric illustration of three potential configurations that placement pegs may take in the system of FIG. 2.

FIG. 4 is an isometric illustration of an additional embodiment of disclosed system for detecting a venous needle dislodgement in the environment of FIG. 1.

FIG. 5A is an isometric illustration of the venous needle dislodgement detector in FIG. 2 including a venous bloodline in an engaged state.

FIG. 5B is an isometric illustration of the venous needle dislodgement detector in FIG. 2 including a venous bloodline in a dislodged state.

FIG. 6 is an isometric illustration of an additional embodiment of a disclosed system for detecting venous needle dislodgement.

FIG. 7A is a diagrammatic illustration of an additional embodiment of a disclosed system for detecting venous needle dislodgement in the environment of FIG. 1.

FIG. 7B is a diagrammatic illustration of additional embodiment in FIG. 6A including a potential venous needle dislodgement.

FIG. 8A is a diagrammatic illustration of an additional embodiment of a disclosed system for detecting venous needle dislodgement in the environment of FIG. 1.

FIG. 8B is a diagrammatic illustration of additional embodiment in FIG. 7A including a potential venous needle dislodgement.

FIG. 9 is a flow chart illustrating an example method that may be performed by the disclosed venous needle dislodgement detection system.

### DETAILED DESCRIPTION

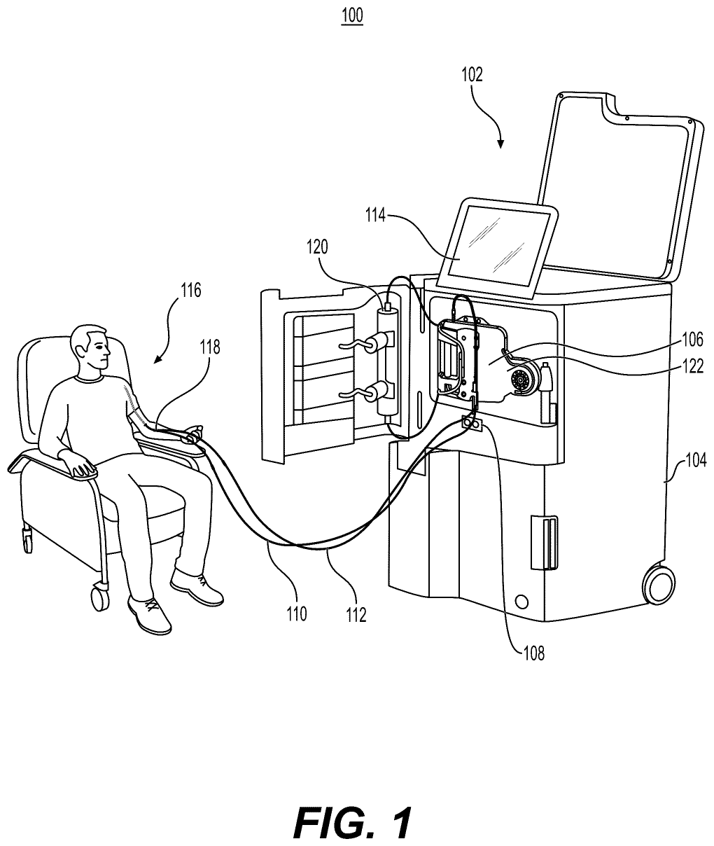

FIG. 1 is an isometric illustration of an exemplary treatment environment 100 (“environment”), including a patient 116, medical blood treatment system 102, and an exemplary disclosed needle dislodgement detector 108 (“VND detector”) for monitoring and automatically halting an ongoing therapy session upon detecting a possible VND event. Environment 100 may be a clinical facility (e.g., a dialysis care center, hospital, or long-term care facility), or in a non-clinical setting, such as at the patient's residence or even onboard a mobile treatment vehicle. While primarily disclosed and illustrated throughout this disclosure in reference to hemodialysis treatment systems, it will be readily apparent to those skilled in the art that the disclosed systems may be effectively deployed to detect VND events during other medical treatments, therapies, or procedures involving an extracorporeal circuit or other direct access to the patient's circulatory system, such as in the case of hemofiltration, apheresis, blood transfusions, plasmapheresis, extracorporcal carbon dioxide removal, extracorporcal cardiopulmonary resuscitation, cardiopulmonary bypass, intravenous feeding, chemotherapy, intravenous antibiotic treatments, etc.

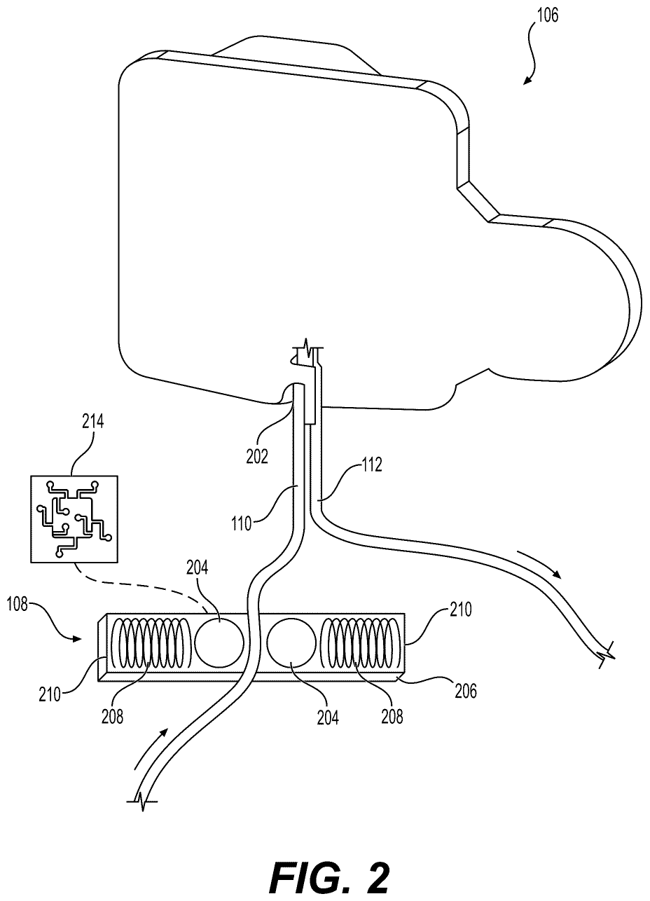

In the example embodiment of FIG. 1, medical blood treatment system 102 is a hemodialysis machine configured to provide dialysis treatment to patient 116. The medical blood treatment system 102 may include a dialysis delivery system (not shown) disposed within a housing 104. A patient blood tubing set including a venous blood line 110 and an arterial blood line 112 may connect the patient's circulatory system to the dialysis delivery system. In some embodiments, medical blood treatment system 102 includes a cartridge 106 that may be removably coupled to a housing 104 prior to therapy. In some embodiments, cartridge 106 may include a dialyzer 120 that is pre-attached to the patient blood tubing set. In other embodiments, cartridge 106 and patient blood tubing set may be connected to dialyzer 120 of the dialysis delivery system. The dialysis delivery system may include a blood pump 122 configured to draw blood from patient 116 through arterial blood line 112, pass the blood through dialyzer 120, and return the treated blood to patient 116 through venous blood line 110. During dialysis therapy, a first end of arterial blood line 112 and a first end 202 (shown in FIG. 2) of venous blood line 110 may be attached to dialyzer 120, a portion of venous blood line 110 extending along a substantially vertical surface of housing 104 proximal to VND detector 108. As discussed above, the dialyzer may form a portion of 106. As illustrated in FIGS. 1-2, cartridge 106 and/or blood tubing set 110-112 may be installed on or within medical blood treatment system 102 such that first end 202 may be considered a fixed anchor point relative to VND detector 108. A remaining portion of venous blood line 110 and arterial blood line 112 may extend from first end 202 (shown in FIG. 2) to patient 116 where the second ends of venous blood line 110 and arterial blood line are inserted at a vascular access 118 (e.g., a fistula) of patient 116 using, for example, a venous needle and an arterial needle to directly access the patient's circulatory system.

Medical blood treatment system 102 may further include a user interface 114 including a display. The treatment status, and other information pertinent to patient treatment, may be accessed and displayed by a user interface at any location or computational scale (e.g., home computer, server, smartphone, the cloud, etc.), or on user interface 114. As illustrated in FIG. 1, user interface 114 may be temporarily or permanently mounted to medical blood treatment system 102. In an alternative embodiment, user interface 114 may be remote from medical blood treatment system 102. For example, user interface 114 may be accessible to patient 116 or another user via wired or wireless communication directly or indirectly including by way of a portal accessible on a remote user interface such as a computer, tablet, or smartphone.

Medical blood treatment system 102 may be provided with a VND detector 108 for monitoring and automatically halting an ongoing therapy session upon detecting a possible VND event. Specifically, VND detector 108 may be configured to monitor the displacement forces and/or movement of venous blood line 110. Upon detecting a threshold magnitude of movement, force, or displacement on venous blood line 110, VND detector 108 may be configured to generate an alarm signal causing medical blood treatment system 102 to halt a treatment session in progress. As illustrated in FIG. 1, VND detector 108 may be mounted to, or form a portion of housing 104 or medical blood treatment system 102. For instance, in FIG. 1, VND detector 108 is illustrated as being mounted to housing 104 substantially vertically below where venous blood line 110 extends from cartridge 106. Other locations and orientations for VND detector 108 will be readily appreciated by one of ordinary skill in the art. VND detector 108 may be placed and oriented so as to detect any motion, displacement, or force exerted on venous blood line 110 indicative of a potential VND event. For example, VND detector 108 may be located vertically above or below cartridge 106, closer to or further away from cartridge 106, or on a different surface of housing 104. Similarly, VND detector 108 may also be located and oriented such that venous blood line 110 extends directly vertically from cartridge 106 to VND detector 108 or may alternatively be positioned and oriented differently depending on the location and orientation of medical blood treatment system 102 as compared to patient 116. VND detector 108 may be fixedly mounted to housing 104 at a specific orientation or may be adjustable depending on the location and orientation of medical blood treatment system 102 relative to patient 116. In one or more embodiments, VND detector 108 may even be mounted to a surface separate from medical blood treatment system 102. As discussed below in reference to FIGS. 2-5, VND detector 108 may take several forms capable of detecting a pulling, tugging, or other displacement force exerted on venous blood line 110 indicative of a potential VND event.

FIG. 2 is an isometric illustration of an exemplary disclosed VND detector 108 within the environment of FIG. 1. In the illustrated embodiment, VND detector 108 may include a controller 214, and a plurality of retention pegs (“pegs”) 204 and a plurality of resilient members 208 set within a guide channel 206 arranged to releasably engage venous blood line 110.

VND detector 108 may be configured to generate a signal indicative of the detected presence of venous blood line 110, enabling medical blood treatment system 102 to begin or continue a therapy session, or to attempt to resume a halted therapy session. VND detector 108 may be configured to monitor and detect displacement or deformation of venous blood line 110 indicative of a potential VND event during operation of medical blood treatment system 102. Upon detecting a displacement or deformation of venous blood line 110 indicative of a potential VND event, VND detector 108 may be configured to generate a signal causing medical blood treatment system 102 to halt a treatment session in progress.

In the embodiment illustrated in FIG. 2, the distance between first end 202 and VND detector 108 is configured to enable VND detector 108 to accurately detect any displacement or deformation of venous blood line 110 indicative of a potential VND event. The effective distance between first end first end 202 and VND detector 108 may vary based various factors including, but not limited to, the mechanical qualities of venous blood line 110; the pressure within venous blood line 110 during a therapy session; and the positions and/or orientations of first end 202, VND detector 108, and/or patient 116 relative to one another. In some instances, venous needle dislodgment may occur under as little as 2.0 pounds of force. Therefore, first end 202 and VND detector 108 should be positioned and configured a sufficient distance apart to detect displacement or deformation of venous blood line 110 under a minimum force required to dislodge a venous needle from patient's 116 access.

In the illustrated embodiment, venous blood line 110 extends in a vertically downward direction from cartridge 106 beginning at first end 202 and continuing through VND detector 108 between pegs 204. One or more of pegs 204 may be located within or along guide channel 206, at least one of pegs 204 being slidably movable along the axis of guide channel 206, the at least one peg 204 being resiliently biased in the direction of opposing peg 204. The illustrated embodiment depicts two pegs 204 each being slidably movable within guide channel 206, and each being resiliently biased inward toward the center of guide channel 206 by resilient members 208, creating a compressive transverse force on either side of venous blood line 110 releasably engage venous blood line 110 between pegs 204. In an alternative embodiment, a first peg 204 may be fixedly attached at a first location along the axis of guide channel 206 while a second peg 204 may be slidably movable within guide channel 206 biased by resilient member 208 in the direction of the first peg 204. In either embodiment, resilient members 208 may be selected to exert sufficient compressive force to retain venous blood line 110 separating pegs 204 while releasing venous blood line 110 in response to a sufficient displacement or deformation force indicative of a potential VND event.

VND detector 108 may be configured to generate a signal indicative of a removal of venous blood line 110 from between pegs 204. In one embodiment, each of pegs 204 may be electrically conductive. In one example embodiment, absent an object such as venous blood line 110 between them, resiliently biased peg(s) 204 traveling along guide channel 206 may be electrically connected forming a closed circuit between them. Upon detecting a closed circuit, controller 214 may generate a signal indicative of the absence of venous blood line 110 separating pegs 204 (a “VND event signal”). Medical blood treatment system 102 may be configured to prevent the start of a therapy session, to halt an ongoing therapy session, and/or to prevent a previously halted therapy session from being resumed in response to receiving a VND event signal. Medical blood treatment system 102 may be further configured to generate one or more audio and/or visual notification(s) and/or instructions regarding the detection of a possible VND event or absence of venous blood line 110 separating pegs 204. For example, controller 214 may be configured to send an audio and/or visual alerts to users via user interface 114 and/or remote user interface(s).

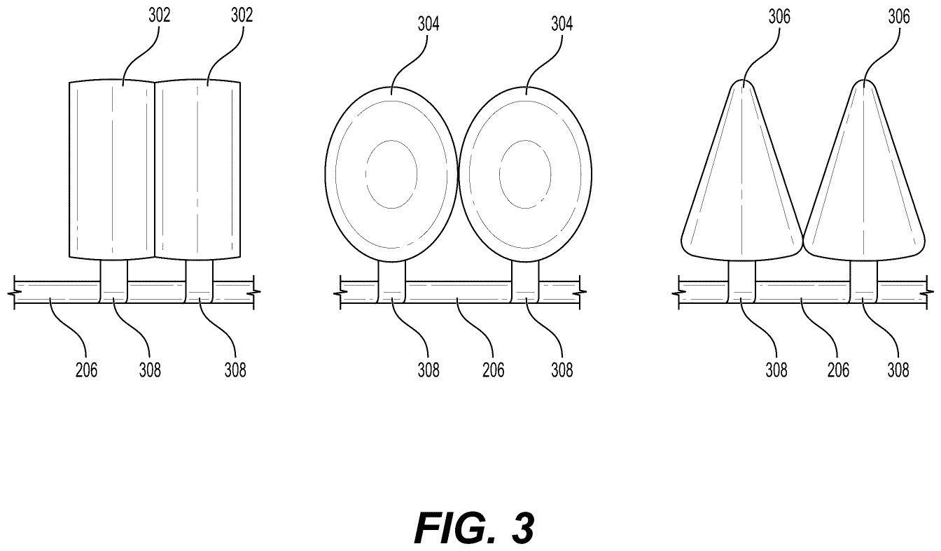

Pegs 204 may extend in a direction normal to guide channel 206 and be of a length sufficient to removably retain venous blood line 110 positioned between them. For example, pegs 204 may extend beyond guide channel 206 at a length equal to the exterior diameter of the venous blood line 110. Alternatively, for greater sensitivity to displacement or deformation forces indicative of a potential VND event, pegs 204 may be shorter. For example, to achieve high sensitivity, pegs 204 may extend only beyond guide channel 206 at a length approximately equal to the exterior radius of the venous blood line 110. The placement pegs 204 may also take the form of different geometric variations discussed below in relation to FIG. 3, below.

VND detector 108 may be a standalone device communicatively coupled (e.g., via wires or wirelessly) to medical blood treatment system 102, or may be a device that is integral with the other components of medical blood treatment system 102. VND detector 108 can include, among other things, a controller 214 including one or more processors, one or more sensors, a memory, and a transceiver. It is contemplated that controller 210 can include additional or fewer components.

The processor of controller 210 may be configured with virtual processing technologies and use logic to simultaneously execute and control any number of operations. The processor may be configured to implement virtual machine or other known technologies to execute, control, run, manipulate, and store any number of software modules, applications, programs, data, etc. In some examples, the processor can be configured to execute instructions to receive commands from medical blood treatment system 102 associated with the status of a treatment session. It is contemplated that, in some examples, controller 210 may be omitted and the disclosed functions may be performed directly by medical blood treatment system 102.

In addition to the open/closed circuit sensor described above, the sensor(s) of VND detector 108 may include one or more of a limit switch or force sensor capable of converting an input mechanical load, weight, tension, compression or pressure into an electrical output signal; and/or an optical sensor such as a camera, through-beam sensor, retro-reflective sensor, LIDAR sensor, a RADAR sensor or the like, configured to detect and/or measure the displacement or deformation of venous blood line 110. A camera can embody one or more semiconductor charge-coupled devices (CCD), complementary metal-oxide-semiconductor (CMOS) devices, and other devices capable of providing digital image data (e.g., video and/or still images) to the associated processor and/or to controller 210. The memory of VND detector 108 can be a volatile or non-volatile memory, removable or non-removable. Some common forms of machine-readable media may include floppy disk, flexible disk, hard disk, magnetic tape, any other magnetic medium, CD-ROM, any other optical medium, punch cards, paper tape, any other physical medium with patterns of holes, ROM, PROM, EPROM, FLASH-EPROM, any other memory chip or cartridge, and/or any other medium from which a processor or computer is adapted to read. Some common forms of volatile memory include SRAM, DRAM, IRAM, and/or any other type of medium which retains its data while devices are powered, potentially losing the memory when the devices are not powered. Some common forms of memory store computer executable code such as firmware that causes the processor of VND detector 108 to perform one or more functions associated with data processing, data storage, data transmitting via the associated sensor, data receiving via the associated sensor, and/or image capture. In some examples, the memory can include one or more buffers for temporarily storing data received from the sensors of VND detector 108, before transmitting the image data to the associated processor and/or to controller 210.

The transceiver of VND detector 108 may include a wired or wireless communication module capable of sending and receiving data via a network (not shown) or via a direct communication link with one or more components in controller 210. In some examples, controller 210 can receive data from medical blood treatment system 102 via the transceiver, including instructions for VND detector 108 to activate the sensors of VND detector 108 and to capture load and/or image data, and for the processor to transmit the image data to medical blood treatment system 102 via the transceiver.

Controller 210 can include one or more processing devices configured to perform functions of the disclosed methods. Controller 210 can constitute a single core or multiple cores executing parallel processes simultaneously. For example, controller 210 can be a single-core processor configured with virtual processing technologies. In certain examples, controller 210 uses logical processors to simultaneously execute and control multiple processes. Controller 210 can implement virtual machine or other known technologies to provide the ability to execute, control, run, manipulate, and store multiple software processes, applications, and programs. In another example, controller 210 includes a multiple-core processor arrangement (e.g., dual core, quad core, etc.) configured to provide parallel processing functionalities that allow simultaneous execution of multiple processes. As discussed in further detail below, controller 210 may be specially configured with one or more applications and/or algorithms for performing method steps and functions of the disclosed examples. It is appreciated that other types of processor arrangements could be implemented that provide for the capabilities disclosed herein.

Controller 210 may include a volatile or non-volatile, magnetic, semiconductor, tape, optical, removable, non-removable, or other type of storage device or tangible and/or non-transitory computer-readable medium that stores one or more executable programs. The computer-readable medium may additionally store data, for example information that is related to a particular treatment session (e.g., a log of detected VND events, detected load/force data, and/or image data).

In some examples, the programs executable by controller 210 include an operating system that performs known functions when executed by controller 210. By way of example, the operating system may include Microsoft Windows™, Unix™, Linux™, Apple™ operating systems, Personal Digital Assistant (PDA) type operating systems such as Microsoft CE™, or another type of operating system. Controller 210 may execute communication software that provides communications with offboard portal 32, such as Web browser software, tablet or smart handheld device networking software, etc.

The programs stored within the computer-readable medium of controller 210 may cause VND detector 108, controller 210, and/or medical blood treatment system 102 to perform processes related to generating, transmitting, storing, receiving, indexing, and/or displaying the force and/or image data collected by the sensors of VND detector 108, as well as information and/or instructions regarding the start of a treatment session, detected VND events, and resuming a halted treatment following a VND event. For example, the programs may be able to configure VND detector 108, controller 210, and/or medical blood treatment system 102 to perform operations including: capturing of the sensor data, displaying a graphical user interface (GUI) for receiving control instructions, receiving control instructions from the associated user via one or more I/O devices and/or the user interface, processing the control instructions, and the like.

FIG. 3 depicts three possible geometric placement peg variations: cylindrical pegs 302, ovoid pegs 304, and conical or tapered pegs 306. Pegs 204 may be configured travel within guide channel 206 the width of pegs 204 or may extend beyond guide channel 206 from peg stalks 308. Pegs 204 and/or peg stalks 308 may be fixedly or removably mounted to sleds or other travel members configured to travel along and be retained within guide channel 206. Alternatively, pegs 204 or peg stalks 308 may be directly configured travel along and be retained within guide channel 206. In one or more embodiments, peg stalks 308 may allow for greater variation in peg geometry. For example, peg stalks 308 may include a threaded portion at a distal end to which pegs 204 may be attached at a similarly threaded cavity configured to receive peg stalks 308. Alternatively, pegs 204 may be configured to rotate freely about the longitudinal axis of peg stalks 308. The displacement or deformation force on venous blood line 110 required to trigger VND event detection may also be adjusted and fine-tuned by selecting different shapes and sizes of pegs 204.

For example, venous blood line 110 may be inserted between pegs 204 such that its lateral sides abut the outer axial surfaces of each of pegs 204. Depending on the orientation and placement of VND detector 108, in response to a displacement force on venous blood line 110 in a direction parallel or slightly oblique to the plane formed by the length of guide channel 206 (such as a vertical downward pull), venous blood line may only slide perpendicular to the cylindrical peg 302 without being fully released from between pegs 204 resulting in a detected VND event. A greater displacement force, or repeated smaller displacement forces may be required to cause venous blood line 110 to be released from between pegs 204 in order to trigger a VND event detection.

Conical pegs 306, by comparison, may be configured to release venous blood line 110 in response to a lower displacement or deformation force. Conical pegs 306 may also allow VND detector 108 to detect and generate alerts, warnings, and or alarms in response to displacement or deformation forces that do not result in the complete release venous blood line 110 from between pegs 204. The tapered shape of conical pegs 306 may also allow for detection of potential VND events resulting from displacement or deformation forces exerted in a direction parallel or slightly oblique to the plane formed by the length of guide channel 206. The smaller the taper of conical pegs 306, the more force may be required for venous blood line 110 to be released from between pegs 204.

Yet more diversity in the sensitivity of VND detector 108 may be achieved by the inclusion of ovoid pegs 304. The relative proportionality of the diameter/volume of ovoid pegs 304 to the exterior diameter of the venous blood line 110 may be adjusted to change the sensitivity of VND detector 108. For example, venous blood line 110 may be held at approximately the exterior diameter of venous blood line 110 by ovoid pegs 304 of approximately the same diameter. For less sensitivity, ovoid pegs 304 may be a higher diameter maintaining contact with venous blood line 110 beyond the exterior diameter, thereby retaining venous blood line 110 nearer to guide channel 206. As a result, small, repeated displacement or deformation forces on venous blood line 110 may not result in a VND event detection. A larger force may be required pull the venous blood line 110 free of ovoid pegs 304.

One skilled in the art will readily recognize that other variations of peg 204 geometry and size may be employed as. For example, pegs 204 may have an ovoid-like end portion proximal to travel guide channel 206, a cylindrical-like center portion, and a conical-like distal end. Material properties and outer surface textures of pegs 204 may also provide for higher or lower coefficients of friction between pegs 204 and venous blood line 110. For example, rubber pegs 204 would be expected to result in a higher coefficient of friction yielding more grip on venous blood line venous blood line 110 than would smooth metal pegs 204.

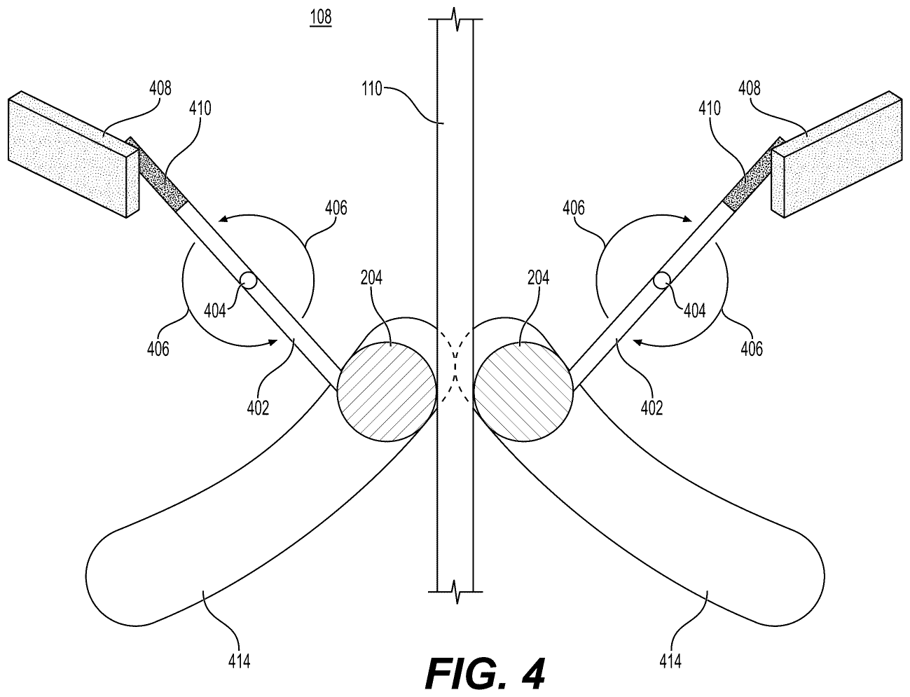

FIG. 4 is a diagrammatic illustration of an alternative embodiment of VND detector 108 configured to releasably engage venous blood line 110 between pegs 204 that are mounted to proximal ends of resiliently biased levers 402. Pegs 204 may protrude a length equivalent to at least one-half the outer diameter of venous blood line 110 in a direction normal to the plane formed by the length of lever 402. Each lever 402 may be resiliently biased axially, being pivotably mounted to VND detector 108 by a pin 404 to rotate or pivot in a direction 406 to releasably engage venous blood line 110 between pegs 204. Lever 402 may include a conductive distal end 410. Lever 402 may rotate in a relaxed state or an engaged state. While in a relaxed state, lever 402 is permitted to rotate in direction 406 such that pegs 204 are proximal to one another and where included, such that conductive distal end 410 abuts sensor plate 408. While in an engaged state, lever 402 is permitted to rotate in direction 406 such that pegs 204 are held apart by releasably engaging venous blood line 110, and where included such that conductive distal end 410 does not sensor plate 408. In a further alternative embodiment, venous blood line 110 may be releasably engaged between one fixed-location peg 204 and one peg 204 mounted to a proximal end of lever 402 resiliently biased in a direction 406. When conductive distal end 410 contacts sensor plate 408, controller 214 may generate a signal indicative of the absence of venous blood line 110 separating pegs 204, and thus a potential VND event.

In one or more embodiments, pegs 204 may be located within or along a curved guide channel 414, and at least one of pegs 204 may be slidably movable along the length of curved guide channel 414. Curved guide channel 414 may have a distal end 416, a proximal end 418, and an upper opening. The shape of curved guide channel 414 may be defined to coincide with the arc produced by peg 204 as lever 402 rotates or pivots in a direction 406 about pin 404. Distal end 416 of curved guide channel 414 may be positioned to allow lever 402 to be rotated opposite direction 406 sufficient to permit venous blood line 110 to be inserted between pegs 204. Proximal end 418 of curved guide channel 414 may be positioned to allow conductive distal end 410 to contact sensor plate 408 when lever 402 is in a relaxed state.

As will be appreciated by a person of skill in the art, lever 402 may be resiliently biased axially to rotate or pivot in a direction 406 by any mechanism known in the art. For example, lever 402 may be resiliently biased by any number of spring members such as a spiral spring or torsion spring coupled with pin 404, or even by selecting pegs 204 composed of magnetic materials sufficient to releasably engage venous blood line 110 between pegs 204. Pegs 204 may be directly mounted to lever 402 or used in conjunction with a peg stalk 308.

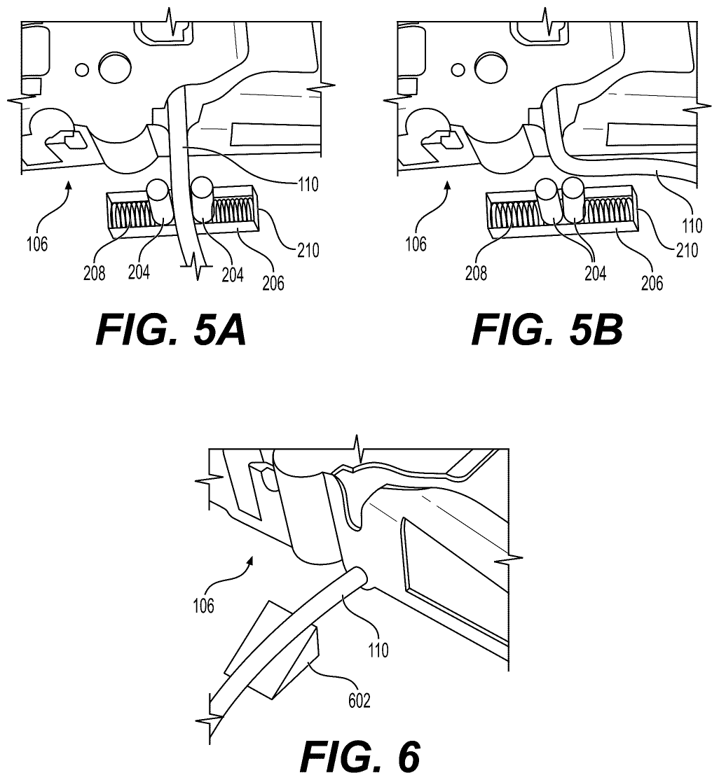

FIG. 5A is an isometric illustration of VND detector 108 as depicted in FIG. 2 including an engaged venous bloodline. Medical blood treatment system 102 may be configured to monitor VND detector 108 prior to and throughout a treatment session to detect a potential VND event. Prior to initiating a treatment session, pegs 204 may be separated within guide channel 206 and venous blood line 110 may be inserted therebetween. Each of pegs 204 may extend beyond guide channel 206 a length sufficient to engage venous blood line 110. Being resiliently biased inward along guide channel 206 by resilient members 208, pegs 204 create a compressive transverse force on either side of venous blood line 110, releasably engaging venous blood line 110 therebetween. Medical blood treatment system 102 may be configured to monitor VND detector 108, and to initiate, continue, and/or resume a treatment session only while venous blood line 110 is releasably engaged between pegs 204.

FIG. 5B is an isometric illustration of VND detector 108 as configured in FIG. 2 including disengaged venous bloodline 110. As a result of a sufficient displacement or deformation force tangential to the longitudinal axis of pegs 204, venous blood line 110 may become completely or at least partially removed or disengaged from between pegs 204. Upon displacement of venous blood line 110 from between pegs 204, VND detector 108 may be configured to generate a VND detection signal indicative of a potential VND event. In response to receiving a VND detection signal, medical blood treatment system 102 may be configured to prevent possible patient blood loss by preventing, halting, or terminating a treatment session.

FIG. 6 depicts a further alternative embodiment of venous VND detector 108 in which guide channel 206, pegs 204, and resilient members 208 are replaced by a limit switch 602. Limit switch 602 may take the form of any commonly available electromechanical device consisting of an actuator mechanically linked to an electrical switch. In FIG. 6, for example, limit switch 602 is depicted as a resiliently pivotable ramp or trigger attached to housing 104 at the vertex of its hypotenuse and base sides. Other configurations and orientations of limit switch 602 are also contemplated. Upon installation and configuration of cartridge 106, venous blood line 110 extends from first end 202 along housing 104, applying sufficient force to depress the perpendicular side of limit switch 602. Medical blood treatment system 102 may be configured to monitor VND detector 108, and to initiate, continue, and/or resume a treatment session only while limit switch 602 remains depressed. When venous blood line 110 is sufficiently displaced or deformed, limit switch 602 is released causing VND detector 108 to generate a VND detection signal.

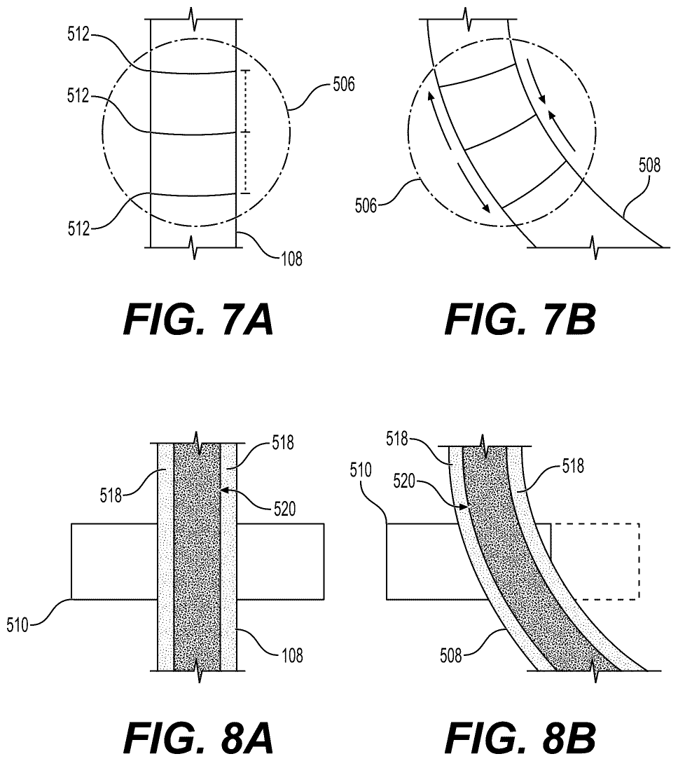

FIGS. 7A-B depict a yet another alternative embodiment of venous VND detector 108 employing an optical sensor 506 to detect displacement or deformation of venous blood line 110. In the illustrated embodiment, venous blood line 110 may be aligned and configured to extend across optical sensor 506. Optical sensor 506 may be configured either to simply detect the presence or absence of venous blood line 110, or alternatively to detect deformation of venous blood line 110. For example, optical sensor 506 may be a contactless presence sensor configured to operate in a reflection mode. For example, optical sensor 506 may be configured to emit and detect light reflected to optical sensor 506 by the tubing of venous blood line 110. In the absence of sufficient light, optical sensor 506 does not detect sufficient light, causing VND detector 108 to generate a VND detection signal.

Alternatively, as shown in FIG. 7A linear indicators 512 may be placed on venous blood line 110 so as to be detectable by optical sensor 506 when venous blood line 110 is at rest. As illustrated in FIG. 7B, optical sensor 506 may be configured to detect a threshold change in distances between linear indicators 512, indicating that venous blood line 110 is bent or otherwise deformed or displaced by a threshold amount, causing VND detector 108 to generate a VND detection signal.

FIGS. 8A-B depict a yet another alternative embodiment of venous VND detector 108 employing an optical sensor with a field of view 510 (“optical sensor area”) to detect a potential VND event. As illustrated in FIG. 8A, optical sensor area 510 may be configured to monitor venous blood line 110 for changes in percent transmittance. Percent transmittance may constitute the percentage of light that can pass through a given material. Optical sensor area 510 may be configured to detect degrees of percent transmittance or may simply detect whether venous blood line 110 crosses optical sensor area 510 as illustrated in FIG. 8B. When venous blood line 110 is at rest, as shown in FIG. 8A, a precent transmittance across optical sensor area 510 should take the form of an inverse bell curve. From left to right, percent transmittance may fall as curvature of the venous blood line 110 increases the thickness of venous blood line wall 518, falls again when passing through returning blood 520, and may increase again across the opposite side of venous blood line wall 518.

### INDUSTRIAL APPLICABILITY

The disclosed system may be applicable to the medical industry in dialysis treatments, or any other type of treatment involving a risk of patient exsanguination in the event that venous blood line 110 becomes dislodged. The disclosed system may automatically detect displacement or deformation forces imparted to venous blood line 110 indicative of a potential VND event, warn of potential VND events, suspend treatment in response to VND events, and resume a halted treatment session upon confirming that venous blood line 110 has been properly realigned. Additionally, the disclosed system may prevent re-initialization of treatments if they have been suspended for a time in excess of safe operational parameters. Operation of the disclosed system is described with reference to FIG. 6.

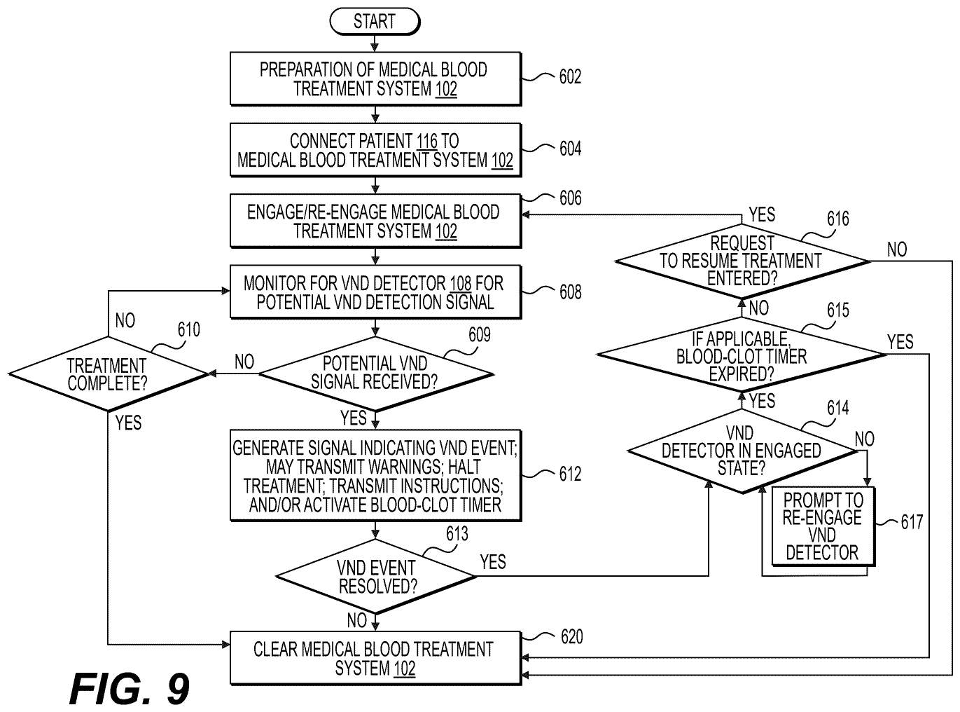

As shown in FIG. 9, the first step in the disclosed system is preparation of medical blood treatment system 102 for treatment (Step 602). Step 602 may include installation and configuration of cartridge 106, and other preparation of medical blood treatment system. Cartridge 106 may allow for the connection of patient blood tubing set to medical blood treatment system 102. In one embodiment, proper configuration of cartridge 106 may be automatically detected through the use of one or more sensors configured to detect proper seating of cartridge 106 and/or connection of patent blood tubing set to medical blood treatment system 102. In another embodiment, the system may be configured to confirm proper configuration of cartridge 106 and connection of patient blood tubing upon receiving an input signal at user interface 114 from the patient 116 or operator. In one or more embodiments, step 602 further comprises automatically preparing and detecting proper configuration of medical blood treatment system 102 to provide treatment. Upon configuring, medical blood treatment system 102, patient 116 may be connected to medical blood treatment system 102 (Step 604)

In Step 604, patient 116 may be connected to medical blood treatment system 102, for example, by cannulation, where venous blood line 110 and arterial blood line 112 inserted into vascular access 116. Commonly, through use of the buttonhole technique, repeated use of a singular insertion location within an arteriovenous fistula, or through the rope ladder technique. At this step venous blood line 110 and arterial blood line 112 may also fastened to patient 116 in order to secure the blood lines by any method known to those performing such treatments including the chevron taping style, butterfly taping style, and/or overlapping taping style.

Upon establishing vascular access 118 using venous blood line 110 and arterial blood line 112, VND detector 108 may be engaged and treatment (cleansing the blood of patient 116) may be started (step 606). The process of engaging VND detector 108 may be determined by the configuration of VND detector 108. For example, the peg-type sensors discussed in reference to FIGS. 2, 4 may be engaged by sliding pegs 204 apart from one another in opposite directions along guide channel 206 and inserting venous blood line 110 therebetween. In the case of a VND detector 108 utilizing a limit switch 602 type sensor, patient 116 or an operator may simply verify that venous blood line 110 is arranged to depress limit switch 602. Alternatively, patient 116 or an operator ensure venous blood line 110 is aligned within optical sensor 506 or optical sensor area 510. Upon detecting venous blood line 110 between pegs 204, or across limit switch 602, optical sensor 506 or optical sensor area 510, controller 214 may generate a signal indicative of VND detector 108 being in an engaged or ready state. The system may then proceed to Step 608.

In Step 608, controller 214 may monitor VND detector 108 for a potential VND detection signal. VND detector 108 may detect a potential displacement or deformation force on venous blood line 110 indicative of a potential VND event according to any of the methods described above with reference to FIGS. 2, 4, 5A-B, 6, 7A-B, or 8A-B. If no alarm conditions occur controller 214 may be configured to determine whether treatment has completed (Step 610). If NO, the system may return to Step 608. Upon receiving a signal from pegs 204, sensor plate 408, limit switch 602, optical sensor 506, or optical sensor area 510 indicating displacement or deformation force on venous blood line 110 the system may proceed to Step 612.

Upon detecting a potential displacement or deformation force on venous blood line 110, VND detector 108 may be configured to generate a signal indicative of a potential VND event (Step 612). In response to receiving a signal indicative of a potential VND event, the disclosed system may be configured to automatically take one or more steps including: (1) transmitting an audio or visual warning to alert patient 116 of the potential VND event; (2) immediately halting treatment, including stopping all components of 102 involved in transmitting blood to or from patient 116, transmitting instructions to user interface 114 with instructions for resolving the potential VND event, and beginning a blood-clot timer. The disclosed system may then return to Step 608 to monitor the status of VND detector 108. Upon determining that VND detector 108 is once again in an engaged state, the system may verify that a blood-clot timer has not expired, and then may resume treatment.

The timer in step 618 may by associated with the accumulation of risk over time that while the treatment has been suspended that returning blood 512 may have begun to coagulate within the venous blood line 110, and/or aspects of medical blood treatment system 102, and/or aspects of cartridge 106. If a time limit imposed by step 618 has not expired, a resume treatment request may be entered.

Resumption of treatment (step 620) may be automatically prevented as discussed in step 618, however a resumption of treatment will require manual confirmation that the venous needle dislodgement event (if any) has been resolved. This may include inspection of the canulation site, adjustment to the site or patient blood tubing set, resetting VND detector 108, and/or any other actions that may be required to safely resume treatment. If the treatment was not resumed within the required amount of time (step 618), or the treatment has ended (step 612), the system must be cleared.

Clearing the system (step 620) will require the removal of patient blood tubing set from medical blood treatment system 102 and/or cartridge 106 (step 614). Additionally, any disposable components of medical blood treatment system 102 and/or cartridge 106 and dialysate materials may be replaced. Treatment information may be updated, and the VND monitoring system may also be reset.

It will be apparent to those skilled in the art that various modifications and variations can be made to the disclosed system. Other embodiments will be apparent to those skilled in the art from consideration of the specification and practice of the disclosed system. It is intended that the specification and examples be considered as exemplary only, with a true scope being indicated by the following claims and their equivalents.

1. A system for detecting dislodgment of a blood access device inserted within a patient during a medical treatment, comprising:
a medical blood access system in communication with a dislodgment sensing apparatus, the medical blood access system comprising:
a venous blood line;
a first end of the venous blood line being coupled to the medical blood access system at a tubing connection point, and
a second end of the venous blood line being coupled to the blood access device;
the dislodgment sensing apparatus comprising a sensor positioned adjacent to the venous blood line and proximal to the tubing connection point, the dislodgment sensing apparatus being configured to:
monitor the medical blood access system; and
generate a dislodgment signal in response to detecting a displacement force imparted to the venous blood line indicative of a potential dislodgment of the blood access device from the patient; and
the medical blood access system being configured to automatically generate an alarm condition in response to receiving the dislodgment signal.

2. The system of claim 1, wherein the dislodgment sensing apparatus further comprises
first and second sensor pegs positioned on each lateral side of the venous blood line;
at least the first sensor peg being movable within a travel channel and biased transverse to the venous blood line towards the second sensor peg, the first sensor peg being separated from the second sensor peg by the venous blood line;
the dislodgment sensing apparatus being configured to generate the dislodgment signal upon removal of the venous blood line from between the first and second sensor pegs.

3. The system of claim 2, wherein the dislodgment sensing apparatus is configured to generate the dislodgement signal upon detecting displacement of the first sensor peg towards the second sensor peg.

4. The system of claim 3, wherein the dislodgment sensing apparatus comprises a limit switch.

5. The system of claim 1, wherein the dislodgment sensing apparatus comprises an optical sensor.

6. The system of claim 5, wherein the optical sensor is further configured to detect a displacement or deformation of the venous blood line by detecting changes in the percent transmittance of the venous blood line, and/or the location thereof.

7. The system of claim 1, wherein the alarm condition generated by the medical blood access system comprises an audible alert indicative of a potential dislodgment of the blood access device from the patient.

8. The system of claim 1, wherein the alarm condition generated by the medical blood access system comprises a visual alert indicative of a potential dislodgment of the blood access device from the patient.

9. The system of claim 2, wherein the medical blood access system is further configured to automatically stop the medical treatment upon receiving the dislodgment signal.

10. The system of claim 9, wherein the medical blood access system is further configured to resume the medical treatment upon replacement of the venous blood line between the first and second sensor pegs.

11. A method for detecting dislodgment of a blood access device inserted within a patient during a medical treatment, comprising:
generating a dislodgment signal in response to detecting a displacement force imparted to the venous blood line indicative of a potential dislodgment of the blood access device from the patient; and
automatically generating an alarm condition in response to receiving the dislodgment signal.

12. The method of claim 11, further comprising indicating to a user a potential venous needle dislodgement event occurred via an audio and/or visual indicator of a sensor detection of forces indicative of a potential venous needle dislodgement event.

13-18. (canceled)

19. The method of claim 12, further comprising stopping treatment by a treatment console, upon sensor detection of the potential venous needle dislodgement event.

20. The method of claim 19, further comprising starting a timer based on actuation of the stoppage of treatment by the treatment console associated with a coagulation potential within the venous blood line or treatment console.

21. The method of claim 20, further comprising preventing resumption of treatment if the timer associated with a coagulation potential within the venous blood line or treatment console has not been addressed before the end of time on said timer.
